(19) United States
(12) Patent Application Publication
(10) Pub. No.: US 2020/0279634 A1
(43) Pub. Date: Sep. 3, 2020

(54) PRE-IMPRINTED MOUTH GUARDS, AND SYSTEMS AND METHODS FOR RETAIL SELECTION OF SAME

(71) Applicant: Guardlab Inc., Farmingdale, NY (US)

(72) Inventors: Aidan Butler, Arcadia, CA (US); Kevin Doyle, Farmingdale, NY (US); Joseph Andary, Burlington (CA); Matthew D. Powell, Georgetown (CA)

(21) Appl. No.: 16/642,823

(22) PCT Filed: May 1, 2018
(86) PCT No.: PCT/CA2018/050518

§ 371 Date: Feb. 27, 2020

Related U.S. Application Data
(60) Provisional application No. 62/492,502, filed on May 1, 2017.
(60) Provisional application No. 62/492,511, filed on May 1, 2017.
(60) Provisional application No. 62/599,653, filed on Dec. 15, 2017.

(51) Int. Cl. G16H 20/40; A61B 5/00; A63B 71/08; G06Q 30/06; G16H 30/40

(52) U.S. Cl. CPC G16H 20/40; A61B 5/0088; G06K 19/0723; G06Q 30/0631; G16H 30/40; A63B 71/085

(57) ABSTRACT

Disclosed herein is mouth guard including a structural component having an arch portion including pre-formed tooth-receiving indentations; and a conforming component supported by the structural component and being displaceable between the teeth of a wearer and the tooth-receiving indentations only when at or above a threshold temperature. Also disclosed is a computer-implemented method for recommending a mouth guard, and a computer-implemented method for determining bite dimensions for each of a plurality of mouth guards.

### CROSS-REFERENCE TO RELATED APPLICATIONS

This application claims priority to U.S. Provisional Patent Application Ser. No. 62/492,502 filed on May 1, 2017 and entitled “Pre-Imprinted Mouth Guard”, to U.S. Provisional Patent Application Ser. No. 62/492,511 filed on May 1, 2017 and entitled “System and Method For Self-Selection of Mouthguard”, and to U.S. Provisional Patent Application Ser. No. 62/599,653 filed on Dec. 15, 2017 and entitled “Pre-Imprinted Mouth Guard.

### FIELD OF THE INVENTION

The following relates generally to mouth guards, and more particularly to mouth guards that can be heated to conform to the bite of a wearer. The following also relates to systems and methods for assisting a customer with choosing a mouth guard, for configuring a custom mouth guard, and for determining bite dimensions for each of a plurality of mouth guards.

### BACKGROUND OF THE INVENTION

Mouth guards are well-known appliances used for impact protection in sports or for ameliorating the effects of medical conditions such as bruxism. Sports mouth guards of the “boil-and-bite” type are made readily available at retail sports outlets, typically in two or three sizes. In order to conform to the mouth of the wearer, some boil and bite mouth guards include a flat plane of material that becomes pliable when heated above a threshold temperature, such as when placed in boiling water. The material, when pliable and when bitten down on by the wearer, is displaced. When cooled, this displaced material re-hardens in its new configuration and thus remains permanently shaped to conform to the bite of the wearer. As such, the pliable material is fully responsible for the fit to the particular wearer's teeth. If re-shaping should be required, the material may be re-heated above the threshold temperature and the fitting process re-done. Because a general-purpose boil and bite mouth guard is designed to account for a wide range of potential mouth sizes and bite configurations, the overall mouth guard size for any one mouth is unlikely to be optimal. In particular, it may be uncomfortable to wear and may not provide the most effective protection on impacts during physical activities for which they are worn. Furthermore, to be able to adapt to a wide range of wearers, a large amount of the mouth guard material is devoted to pliability rather than to actual support.

Custom mouth guards for sports or other activities are also available. One or more digital scans of the wearer's mouth can be acquired using specialized imaging equipment, and processing is conducted on the digital scans to generate a digital model of the wearer's teeth and bite. Based on the digital model, a counterpart custom mouth guard can be constructed, with the mouth guard having indentations for each of the actual wearer's teeth and that support the particular wearer's bite. Optionally, the wearer can choose the style and structure of mouth guard based on the needs of the particular sport or other activity for which the mouth guard is to be worn. Since the fit is customized to the wearer, with custom mouth guards there is no need for pliable material that is meant to conform post-delivery to teeth. While custom mouth guards provide generally a superior fit than the more generic boil-and-bite mouth guards, and are more supportive because they can be made with sufficient structure and still conform well to a particular wearer's mouth, the process of acquiring digital scans, generating a digital model and forming the custom mouth guard can be time-consuming and expensive. Accordingly, only serious athletes or those motivated by particular medical issues such as bruxism are inclined to undertake the effort and expense of having a custom mouth guard made for them.

### SUMMARY OF THE INVENTION

In accordance with an aspect, there is provided a mouth guard comprising a structural component having an arch portion including pre-formed tooth-receiving indentations; and a conforming component supported by the structural component and being displaceable between the teeth of a wearer and the tooth-receiving indentations only when at or above a threshold temperature.

In an embodiment, the conforming component is adapted to the pre-formed tooth-receiving indentations, so as to enter into and partially fill the tooth-receiving indentations. In an alternative embodiment, the conforming component is a generally flat layer of material overlying the pre-formed tooth-receiving indentations.

In an embodiment, at least the conforming component is formed as a plurality of adjacent substantially planar surfaces intersecting along respective lines, wherein the lines soften and the surfaces wrap around a wearer's teeth when subjected to pressure by the teeth at or above the threshold temperature, thereby to conform to the wearer's teeth.

Because the structural component has pre-formed tooth-receiving indentations, the fit to, and support of, a wearer's teeth can be shared between the structural component and the conforming component. The conforming component may be thought of as filling in any gaps between the pre-formed tooth-receiving indentations and the wearer's teeth, so as to fine-tune the fit, rather than forming the fit anew and thus being wholly responsible for the fit. Accordingly, the amount of displacement required of the conforming component is less than prior art boil and bite mouth guards. This, in turn, enables relatively more material in the mouth guard to be devoted to structure and support than known generic boil and bite devices.

In accordance with another aspect, there is provided a computer-implemented method for recommending a mouth guard, the method comprising receiving electronic data including an individual bite profile comprising bite dimensions for an individual; automatically selecting, from a set of different pre-fabricated mouth guards, at least one pre-fabricated mouth guard with a mouth guard profile most closely corresponding to the individual bite profile; and generating an output message recommending the selected at least one pre-fabricated mouth guard.

In an embodiment, the individual bite profile data is derived from an electronic scan of the individual's mouth. In embodiment, the individual bite profile data is derived from one or more digital images of the individual's mouth. In an embodiment, the individual bite profile data is derived from one or more digital images of bite paper used by the individual. In an embodiment, the individual bite profile data is derived from one or more digital videos of the individual's mouth.

In an embodiment, the mouth guard profile for each of the pre-fabricated mouth guards in the set comprises a range of potential bite dimensions, wherein automatically selecting comprises calculating, using actual bite dimensions from the individual bite profile, within which of the ranges of potential bite dimensions the actual bite dimensions lies.

In an embodiment, the mouth guard profile for each of the pre-fabricated mouth guards in the set comprises representative bite dimensions, wherein automatically selecting comprises calculating, using actual bite dimensions from the individual bite profile, which of the representative bite dimensions differs the least from the actual bite dimensions.

In an embodiment, the method further includes providing an application for a mobile device, the application enabling a user to capture the one or more digital videos and provide the one or more digital videos for the deriving.

In an embodiment, the application comprises a user interface for aiding orientation of a digital video camera of the mobile device with respect to the mouth of the individual. In an embodiment, the user interface comprises an augmented reality component for aiding the orientation of the digital video camera.

In accordance with another aspect, there is provided a computer-implemented method for determining bite dimensions for each of a plurality of mouth guards, the method comprising maintaining a plurality of individual bite profiles each comprising bite dimensions for respective individuals; clustering the individual bite profiles into a predetermined number of clusters according to similarities between respective bite dimensions; and for each of the clusters, generating, based on the bite dimensions of the individual bite profiles in the cluster, a mouth guard profile comprising at least one of: a unique range of potential bite dimensions to be accommodated by a respective mouth guard; and unique representative bite dimensions for the respective mouth guard.

In an embodiment, the predetermined number of clusters is at least four. In an embodiment, the predetermined number of clusters is ten.

According to another aspect, there is provided a processor-readable medium embodying a non-transitory computer program for recommending a mouth guard, the computer program comprising program code for receiving electronic data including an individual bite profile comprising bite dimensions for an individual; program code for automatically selecting, from a set of different pre-fabricated mouth guards, at least one pre-fabricated mouth guard with a mouth guard profile most closely corresponding to the individual bite profile; and program code for generating an output message recommending the selected at least one pre-fabricated mouth guard.

According to another aspect, there is provided a computer-readable medium embodying a non-transitory computer program for determining bite dimensions for each of a plurality of mouth guards, the computer program comprising program code for maintaining a plurality of individual bite profiles each comprising bite dimensions for respective individuals; program code for clustering the individual bite profiles into a predetermined number of clusters according to similarities between respective bite dimensions; and program code for, for each of the clusters, generating, based on the bite dimensions of the individual bite profiles in the cluster, at least one of: a unique range of potential bite dimensions to be accommodated by a respective mouth guard; and unique representative bite dimensions for the respective mouth guard.

Other aspects and embodiments are disclosed herein.

### BRIEF DESCRIPTION OF THE DRAWINGS

Embodiments of the invention will now be described with reference to the appended drawings in which:

FIG. 1 is a front perspective view of a mouth guard, according to an embodiment;

FIG. 2 is a top plan view of the mouth guard of FIG. 1;

FIG. 3 is a front perspective view of an alternative mouth guard, according to an embodiment;

FIG. 4 is a rear perspective view of digital model of a device for forming tooth-receiving indentations for an alternative mouth guard;

FIG. 5 is a front perspective view of the digital model of FIG. 4;

FIG. 6 is a front elevational view of the digital model of FIG. 4;

FIG. 7 is a side perspective view of the device for forming tooth receiving indentations depicted by the digital model of FIG. 4;

FIG. 8 is a top plan view of a mouth guard having tooth-receiving indentations formed using the device of FIG. 7;

FIG. 9 is a side elevational view of another mouth guard, according to an embodiment;

FIG. 10 is a top plan view of two prior art “boil and bite” mouth guards;

FIG. 11 is a bottom plan view of another mouth guard according to an embodiment;

FIG. 12A is a flowchart depicting steps in a computer-implemented method for recommending a mouth guard, according to an embodiment;

FIG. 12B is a screen shot of a user interface for an application running on a mobile device for orienting a digital camera of the mobile device correctly with respect to the mouth of an individual for whom a mouth guard is being selected;

FIG. 13 is a block diagram of a special purpose computing system on which computer-implemented methods described in the application may be executed; and

FIG. 14 is a flowchart depicting steps in a computer-implemented method for determining bite dimensions for each of a plurality of mouth guards, according to an embodiment.

### DETAILED DESCRIPTION

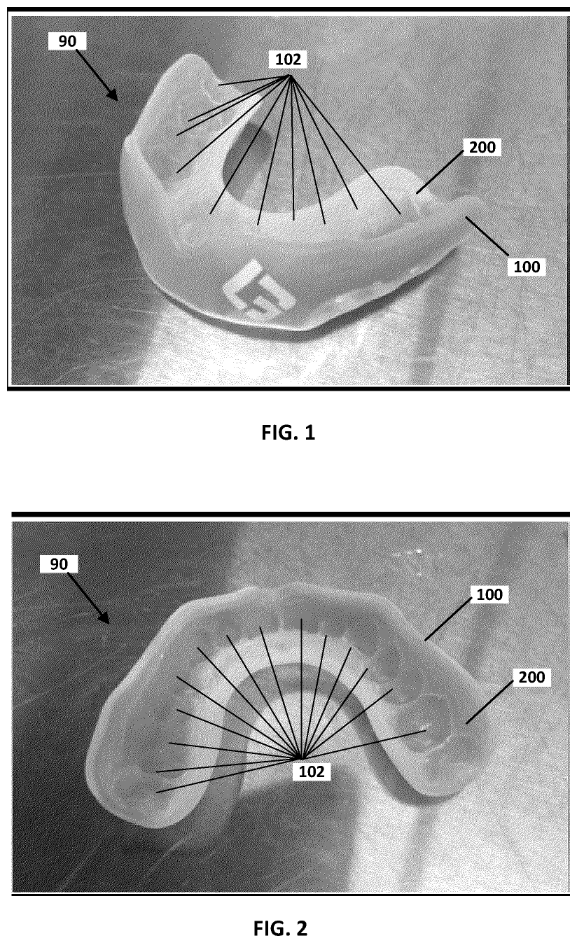

FIG. 1 is a front perspective view of a mouth guard 90, according to an embodiment. FIG. 2 is a top plan view of mouth guard 90. Mouth guard 90 includes a structural component 100 having an arch portion that includes pre-formed tooth-receiving indentations 102. A conforming component 200 is supported by the structural component 100 and is displaceable between the teeth of a wearer and the tooth-receiving indentations 102 only when at or above a threshold temperature. In the embodiment shown in FIGS. 1 and 2, the conforming component 200 is substantially transparent such that the tooth-receiving indentations 102 can be seen through the conforming component 200. Furthermore, in the embodiment shown in FIGS. 1 and 2, the conforming component is adapted to the pre-formed tooth-receiving indentations such that the conforming component partially fills the pre-formed tooth-receiving indentations. As a result, the mouth guard 90 appears shaped somewhat like a custom mouth guard.

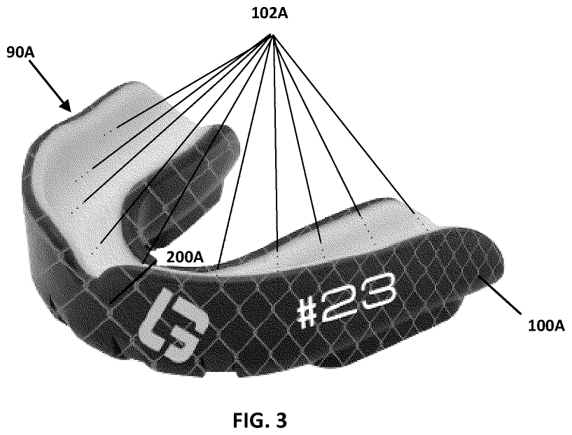

An alternative mouth guard 90A is shown in FIG. 3. In this embodiment, conforming component 200A is generally flat layer of material overlying pre-formed tooth-receiving indentations 102A. In this embodiment, the material of conforming component 200A is opaque such that the pre-formed tooth-receiving indentations are not generally visible through the conforming component 200A. However, when heated and bitten into, the conforming component 200A is similarly displaceable between the teeth of the wearer and the tooth-receiving indentations 102. Other configurations, such as those in which the conforming component 200A is a flat layer but is not opaque, may be provided.

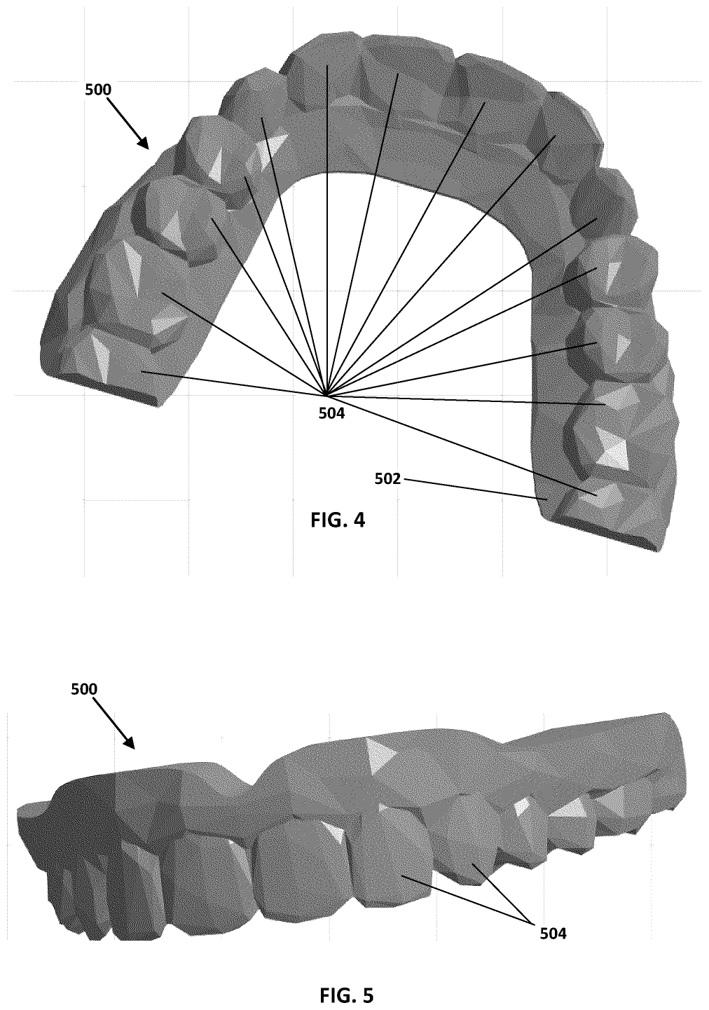

Alternative mouth guards are contemplated herein, as are devices and method for forming such alternative mouth guards. For example, FIG. 4 is a rear perspective view of digital model of a device 500 for forming tooth-receiving indentations for an alternative mouth guard. In turn, FIG. 5 is a front perspective view of the digital model of FIG. 4, and FIG. 6 is a front elevational view of the digital model of FIG. 4.

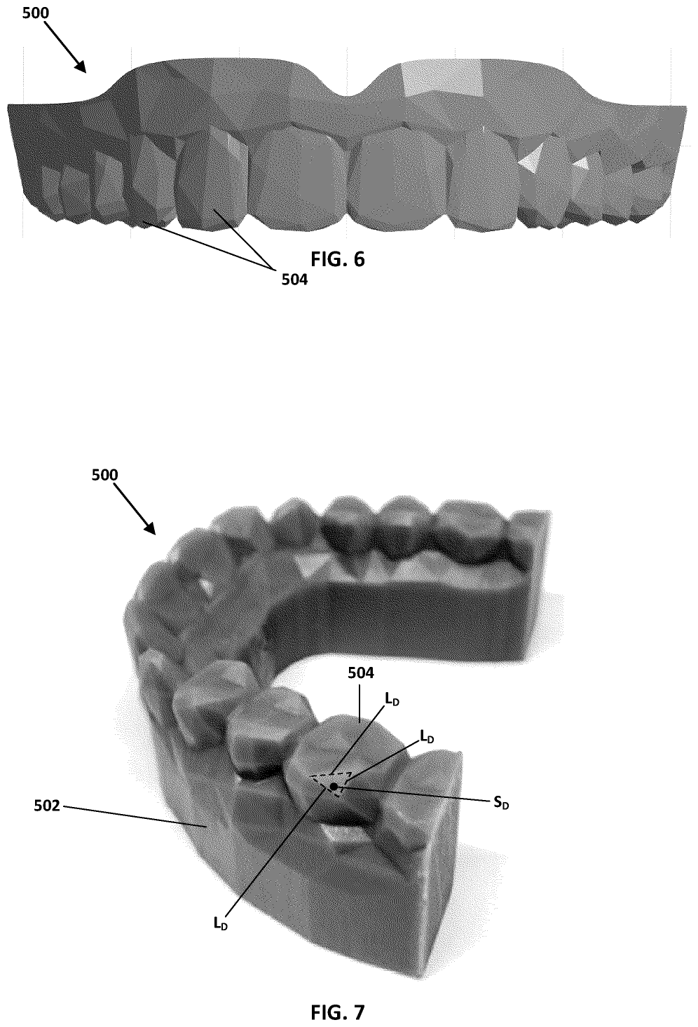

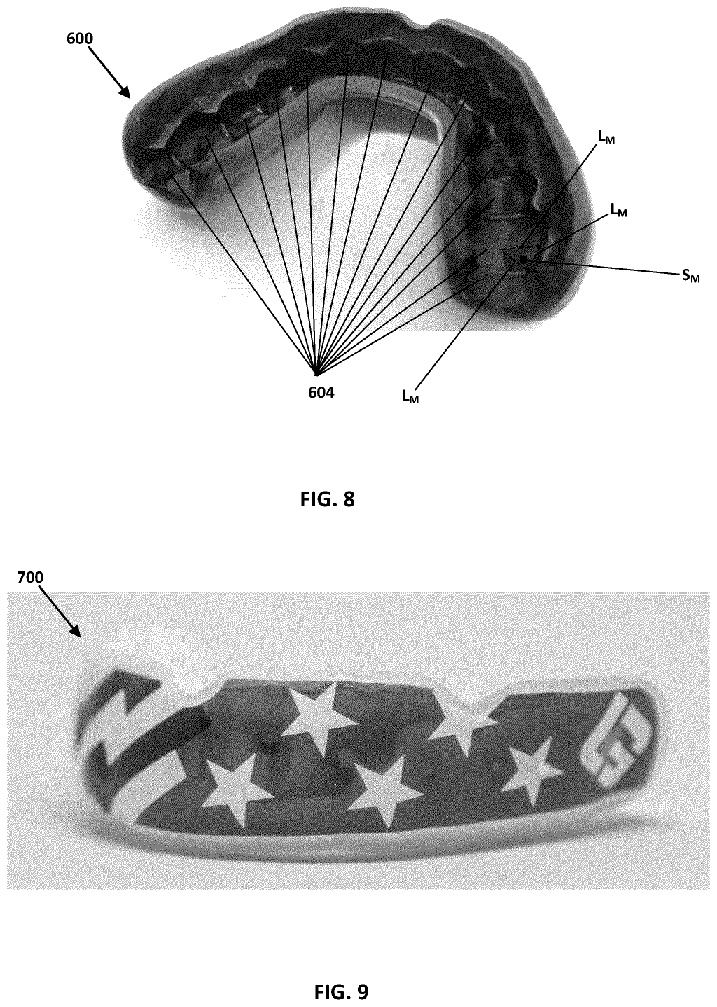

FIG. 7 is a side perspective view of the device 500 depicted by the digital model of FIGS. 4 through 6. As shown in FIGS. 6 and 7, device 500 includes a base 502 and a plurality of individually-sized and shaped projections 504 for forming corresponding indentations 604 of a mouth guard, such as mouth guard 600 shown in FIG. 8. Projections 504 are not all the same shape and size, as they are each intended to serve as generalized, or rough, representations of individual teeth.

Each projection 504 includes a respective set of interfacing, polygonal-shaped surfaces SD (only some of which are marked as such). The surfaces SD interface with adjacent surfaces SD at their periphery at particular angles, and the lines of intersection form counterpart lines of intersections L_{D} (only some of which are marked as such) in the device 500. These form corresponding surfaces S_{M} in the indentations 604 of mouth guard 600 formed by projections 504. The lines of intersection L_{M} correspondingly formed in the indentations 604 of mouth guard 600 are sharp and well-defined, but when the material forming the conforming component (being so shaped by device 500 and in particular projections 504) is heated and subjected to pressure from interaction with a wearer's teeth, the material deforms such that the lines of intersection L_{D} “soften” and the somewhat-planar surfaces SD “wrap” around the wearer's teeth when displaced under heat and pressure. When the material forming the conforming component is subsequently cooled, the softened lines L_{D} and wrapped surfaces SD remain where they have settled against the teeth, so that each time the mouth guard 600 is worn, the wearer's teeth are supported closely within the mouth guard 600.

FIG. 9 is a side elevational view of another mouth guard 700 according to embodiments of the invention.

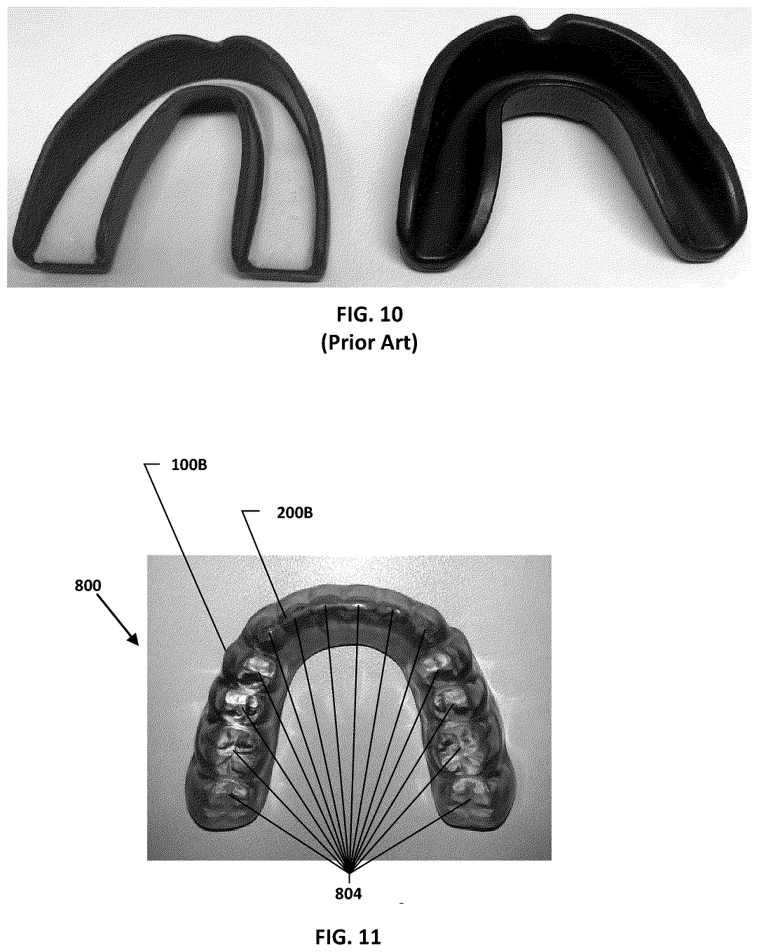

FIG. 10 is a top plan view of two prior art “boil and bite” mouth guards. It will be noted that such prior art “boil and bite” mouth guards do not include individual tooth-receiving indentations and thus do not provide as close as close a match to a wearer's teeth as do embodiments of the present invention disclosed herein.

FIG. 11 is a bottom plan view of another mouth guard 800 according to an embodiment. Mouth Guard 800 is suitable for night time sleep for ameliorating the effects of bruxism, and includes indentations 804 in pliable material 200B supported within a support structure 100B of less pliable material. Mouth guard 800 was initially formed with indentations 804 that correspond to individually-sized and shaped projections 504 having respective sets of interfacing, polygonal-shaped surfaces similar to those provided by device 500, and was subjected to the boil-and-bite process to cause the lines of intersection to soften and the surfaces themselves to wrap around the wearer's teeth thereby to arrive at the form and condition shown in FIG. 11.

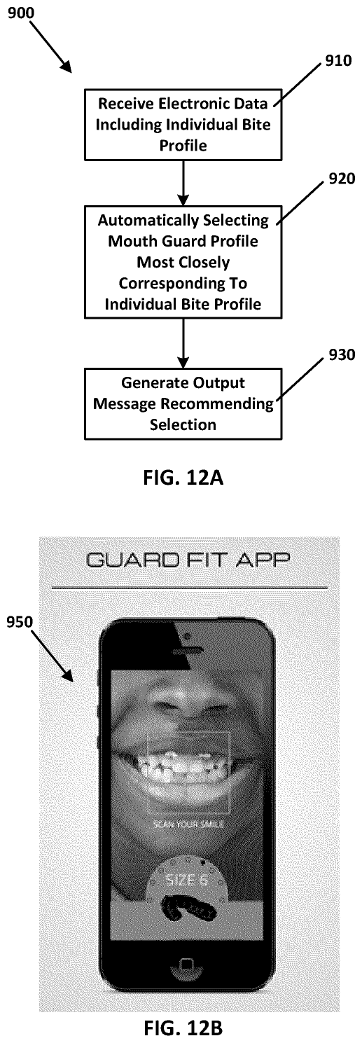

It is useful to provide a range of dimensions of mouth guards such as those disclosed here in a retail environment, whether it be at a bricks-and-mortar store, or online. Such a range would include mouth guards having mouth guard profiles including respective arch dimensions and other features corresponding to the intended use, such as additional support for body contact sports or other features for night time sleeping. Furthermore, if a range includes four or more choices of mouth guard dimensions for an individual, it is useful to provide some retail-level assistance as to which mouth guard dimensions are best-suited to the individual for whom the mouth guard is being purchased. FIG. 12A is a flowchart depicting steps in a computer-implemented method 900 for recommending a mouth guard. During the method, electronic data including an individual bite profile comprising bite dimensions for an individual is received (step 910). At least one pre-fabricated mouth guard with a mouth guard profile most closely corresponding to the individual bite profile is automatically selected from a set of different pre-fabricated mouth guards (step 920). Then, an output message is generated recommending the selected at least one pre-fabricated mouth guard (step 930).

The computer-implemented method may be executed in whole or in part on a mobile device 950 such as is shown in FIG. 12B, for example as part of a retail transaction involving an online purchase from an e-commerce retailer such as Amazon.com. The computer-implemented method may be executed in whole or in part on a retail kiosk, for example as part of a retail transaction involving an in-store purchase from a sports store (most typically for a sports mouth guard) or a from pharmacy (most typically for a mouth guard for ameliorating bruxism).

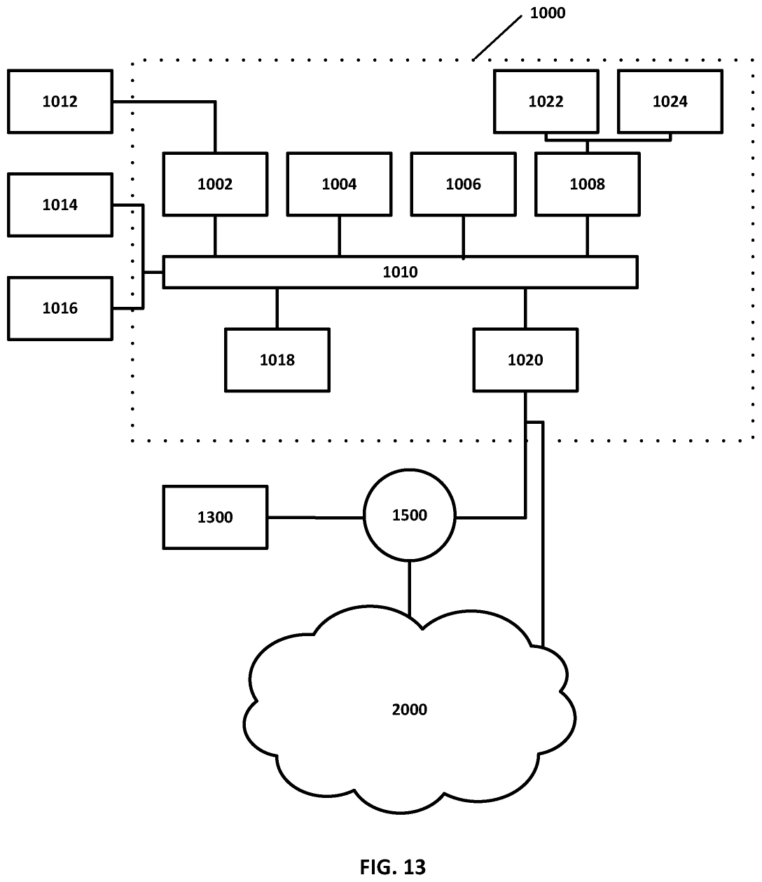

More generally, process 900 is executed on one or more systems such as special purpose computing system 1000 shown in FIG. 13. Computing system 1000 may also be specially configured with software applications and hardware components to enable a user to capture and play media such as digital video, as well as to encode, decode and/or transcode the digital video from and into various formats such as MP4, AVI, MOV, WEBM and using a selected compression algorithm such as H.264 or H.265 and according to various selected parameters, thereby to compress, decompress, view and/or manipulate the digital video as desired for a particular application, media player, or platform. Computing system 1000 may also be configured to enable an author or editor to form multiple copies of a particular digital video, each encoded with a respective bitrate, to facilitate streaming of the same digital video to various downstream systems.

Computing system 1000 includes a bus 1010 or other communication mechanism for communicating information, and a processor 1018 coupled with the bus 1010 for processing the information. The computing system 1000 also includes a main memory 1004, such as a random access memory (RAM) or other dynamic storage device (e.g., dynamic RAM (DRAM), static RAM (SRAM), and synchronous DRAM (SDRAM)), coupled to the bus 1010 for storing information and instructions to be executed by processor 1018. In addition, the main memory 1004 may be used for storing temporary variables or other intermediate information during the execution of instructions by the processor 1018. Processor 1018 may include memory structures such as registers for storing such temporary variables or other intermediate information during execution of instructions. The computing system 1000 further includes a read only memory (ROM) 1006 or other static storage device (e.g., programmable ROM (PROM), erasable PROM (EPROM), and electrically erasable PROM (EEPROM)) coupled to the bus 1010 for storing static information and instructions for the processor 1018.

The computing system 1000 also includes a disk controller 1008 coupled to the bus 1010 to control one or more storage devices for storing information and instructions, such as a magnetic hard disk 1022 and/or a solid state drive (SSD) and/or a flash drive, and a removable media drive 1024 (e.g., solid state drive such as USB key or external hard drive, floppy disk drive, read-only compact disc drive, read/write compact disc drive, compact disc jukebox, tape drive, and removable magneto-optical drive). The storage devices may be added to the computing system 1000 using an appropriate device interface (e.g., Serial ATA (SATA), peripheral component interconnect (PCI), small computing system interface (SCSI), integrated device electronics (IDE), enhanced-IDE (E-IDE), direct memory access (DMA), ultra-DMA, as well as cloud-based device interfaces).

The computing system 1000 may also include special purpose logic devices (e.g., application specific integrated circuits (ASICs)) or configurable logic devices (e.g., simple programmable logic devices (SPLDs), complex programmable logic devices (CPLDs), and field programmable gate arrays (FPGAs)).

The computing system 1000 also includes a display controller 1002 coupled to the bus 1010 to control a display 1012, such as an LED (light emitting diode) screen, organic LED (OLED) screen, liquid crystal display (LCD) screen or some other device suitable for displaying information to a computer user. In an embodiment, display controller 1002 may incorporate a dedicated graphics processing unit (GPU) for processing mainly graphics-intensive or other highly-parallel operations. Such operations may include rendering by applying texturing, shading and the like to wireframe objects including polygons such as spheres and cubes thereby to relieve processor 1018 of having to undertake such intensive operations at the expense of overall performance of computing system 1000, thereby to undertake certain augmented reality functionality or the like. A GPU may incorporate dedicated graphics memory for storing data generated during its operations, and includes a frame buffer RAM memory for storing processing results as bitmaps to be used to activate pixels of display 1012. A GPU may be instructed to undertake various operations by applications running on computing system 1000 using a graphics-directed application programming interface (API) such as OpenGL, Direct3D and the like.

The computing system 1000 includes input devices, such as for example a keyboard 1014 and a pointing device 1016, for interacting with a computer user and providing information to the processor 1018. The pointing device 1016, for example, may be a mouse, a trackball, or a pointing stick for communicating direction information and command selections to the processor 1018 and for controlling cursor movement on the display 1012. The computing system 1000 may employ a display device that is coupled with an input device, such as a touch screen. Other input devices may be employed, such as those that provide data to the computing system via wires or wirelessly, such as gesture detectors including infrared detectors, gyroscopes, accelerometers, radar/sonar and the like. A printer may provide printed listings of data stored and/or generated by the computing system 1000.

The computing system 1000 performs a portion or all of the processing steps discussed herein in response to the processor 1018 and/or GPU of display controller 1002 executing one or more sequences of one or more instructions contained in a memory, such as the main memory 1004. Such instructions may be read into the main memory 1004 from another processor readable medium, such as a hard disk 1022 or a removable media drive 1024. One or more processors in a multi-processing arrangement such as computing system 1000 having both a central processing unit and one or more graphics processing unit may also be employed to execute the sequences of instructions contained in main memory 1004 or in dedicated graphics memory of the GPU. In alternative embodiments, hard-wired circuitry may be used in place of or in combination with software instructions.

As stated above, the computing system 1000 includes at least one processor readable medium or memory for holding instructions programmed according to the teachings of the invention and for containing data structures, tables, records, or other data described herein. Examples of processor readable media are solid state devices (SSD), flash-based drives, compact discs, hard disks, floppy disks, tape, magneto-optical disks, PROMs (EPROM, EEPROM, flash EPROM), DRAM, SRAM, SDRAM, or any other magnetic medium, compact discs (e.g., CD-ROM), or any other optical medium, punch cards, paper tape, or other physical medium with patterns of holes, a carrier wave (described below), or any other medium from which a computer can read.

Stored on any one or on a combination of processor readable media, includes software for controlling the computing system 1000, for driving a device or devices to perform the functions discussed herein, and for enabling the computing system 1000 to interact with a human user (e.g., customer). Such software may include, but is not limited to, device drivers, operating systems, development tools, and applications software. Such processor readable media further includes the computer program product for performing all or a portion (if processing is distributed) of the processing performed discussed herein.

The computer code devices of discussed herein may be any interpretable or executable code mechanism, including but not limited to scripts, interpretable programs, dynamic link libraries (DLLs), Java classes, and complete executable programs. Moreover, parts of the processing of the present invention may be distributed for better performance, reliability, and/or cost.

A processor readable medium providing instructions to a processor 1018 may take many forms, including but not limited to, non-volatile media, volatile media, and transmission media. Non-volatile media includes, for example, optical, magnetic disks, and magneto-optical disks, such as the hard disk 1022 or the removable media drive 1024. Volatile media includes dynamic memory, such as the main memory 1004. Transmission media includes coaxial cables, copper wire and fiber optics, including the wires that make up the bus 1010. Transmission media also may also take the form of acoustic or light waves, such as those generated during radio wave and infrared data communications using various communications protocols.

Various forms of processor readable media may be involved in carrying out one or more sequences of one or more instructions to processor 1018 for execution. For example, the instructions may initially be carried on a magnetic disk of a remote computer. The remote computer can load the instructions for implementing all or a portion of the present invention remotely into a dynamic memory and send the instructions over a wired or wireless connection using a modem. A modem local to the computing system 1000 may receive the data via wired Ethernet or wirelessly via WiFi and place the data on the bus 1010. The bus 1010 carries the data to the main memory 1004, from which the processor 1018 retrieves and executes the instructions. The instructions received by the main memory 1004 may optionally be stored on storage device 1022 or 1024 either before or after execution by processor 1018.

The computing system 1000 also includes a communication interface 1020 coupled to the bus 1010. The communication interface 1020 provides a two-way data communication coupling to a network link that is connected to, for example, a local area network (LAN) 1500, or to another communications network 2000 such as the Internet. For example, the communication interface 1020 may be a network interface card to attach to any packet switched LAN. As another example, the communication interface 1020 may be an asymmetrical digital subscriber line (ADSL) card, an integrated services digital network (ISDN) card or a modem to provide a data communication connection to a corresponding type of communications line. Wireless links may also be implemented. In any such implementation, the communication interface 1020 sends and receives electrical, electromagnetic or optical signals that carry digital data streams representing various types of information.

The network link typically provides data communication through one or more networks to other data devices, including without limitation to enable the flow of electronic information. For example, the network link may provide a connection to another computer through a local network 1500 (e.g., a LAN) or through equipment operated by a service provider, which provides communication services through a communications network 2000. The local network 1500 and the communications network 2000 use, for example, electrical, electromagnetic, or optical signals that carry digital data streams, and the associated physical layer (e.g., CAT 5 cable, coaxial cable, optical fiber, etc). The signals through the various networks and the signals on the network link and through the communication interface 1020, which carry the digital data to and from the computing system 1000, may be implemented in baseband signals, or carrier wave based signals. The baseband signals convey the digital data as unmodulated electrical pulses that are descriptive of a stream of digital data bits, where the term “bits” is to be construed broadly to mean symbol, where each symbol conveys at least one or more information bits. The digital data may also be used to modulate a carrier wave, such as with amplitude, phase and/or frequency shift keyed signals that are propagated over a conductive media, or transmitted as electromagnetic waves through a propagation medium. Thus, the digital data may be sent as unmodulated baseband data through a “wired” communication channel and/or sent within a predetermined frequency band, different than baseband, by modulating a carrier wave. The computing system 1000 can transmit and receive data, including program code, through the network(s) 1500 and 2000, the network link and the communication interface 1020. Moreover, the network link may provide a connection through a LAN 1500 to a mobile device 1300 such as a personal digital assistant (PDA) laptop computer, or cellular telephone or smartphone.

The electronic data store implemented in the database described herein may be one or more of a table, an array, a database, a structured data file, an XML file, or some other functional data store, such as hard disk 1022 or removable media 1024.

Measurement of the arch size of a customer can be done in various ways. For example, the open mouth of a customer in a retail environment can be digitally photographed from one or more vantage points using a device in a self-serve kiosk. This could capture upper and lower arch dimension information, including distance from cuspid tip to cuspid tip in the lower arch to cross reference for accurate mouth guard recommendations. Alternatively, the customer can self-photograph using a smartphone or other similar device along with a token used as a size reference, such as a particular coin. The captured digital image along with the size reference token can then be processed in order to gauge the arch size, and the arch size cross-referenced against the pre-established subranges of available sizes to determine in which subrange the customer's arch size falls. The mouth guard size corresponding to the subrange can then be determined and recommended to the customer. For example, using a mobile application, the customer can identify his or her arch size, can select the appropriate mouth guard size accordingly, and can then submit an actual order for delivery or a reservation for pickup from a local retail outlet. Using a self-service kiosk within a retail outlet, the customer can identify the appropriate size and then simply find it on the shelf or display case.

Other methods for measuring arch size can be used. For example, a retail kiosk may be provided with a small convenient dispenser for dispensing “bite paper” which the customer can bite down on and that registers the bite as indentations on the bite paper. The customer can then provide the indented bite paper to the kiosk for digital imaging and processing to automatically recommend a size, or the bite paper itself can have a visual scale with which the customer can determine arch size and, consequently mouth guard size, directly.

Therefore, in embodiments, the individual bite profile data can be derived from an electronic scan of the individual's mouth, from one or more digital images of the individual's mouth, from one or more digital images of bite paper made available most typically in a retail kiosk environment and used by the individual, and/or from one or more digital videos of the individual's mouth. Such derivations may include various edge, pattern and/or object detection as is known in the field of digital image processing.

Where digital videos or digital images are concerned, an application for a mobile device may be provided that enables a user to capture the one or more digital videos and/or one or more digital images and provide the one or more digital videos or the digital images for the deriving.

In an embodiment, such an application comprises a user interface for aiding orientation of a digital video camera of the mobile device with respect to the mouth of the individual. For example, a target box may be displayed as a region of interest on a screen of the mobile device and a user is to orient the mobile device digital video camera so as to capture the mouth of the individual for whom the mouth guard is being selected within the region of interest, such as is shown in FIG. 12B. In another embodiment, the user interface comprises an augmented reality component displaying graphics or other user interface elements such as graphical teeth or graphical grins positioned in real-time atop the display of the actual teeth/grins being captured by the digital camera and displayed on the device, for aiding a user thereby easing the process of correctly orienting the digital video camera.

There are various processes available for automatically selecting the appropriate mouth guard using the individual bite profile data that is received. For example, where the mouth guard profile for each of the pre-fabricated mouth guards in the set comprises a range of potential bite dimensions (representing respective dentitions: arch size, tooth sizes), the automatically selecting may include calculating, using actual bite dimensions (arch size, tooth sizes) from the individual bite profile, within which of the ranges of potential bite dimensions the actual bite dimensions lies.

As another example, where the mouth guard profile for each of the pre-fabricated mouth guards in the set comprises representative bite dimensions such as bite dimensions representing a single dentition (arch size, tooth sizes), wherein automatically selecting comprises calculating, using actual bite dimensions (arch size, tooth sizes) from the individual bite profile, which of the representative bite dimensions differs the least from the actual bite dimensions. This may be done by calculating which mouth guard profile comprises representative bite dimensions that provide the best least-squares fit with the actual bite dimensions of the individual bite profile.

Alternatives for automatically selecting a mouth guard profile most closely corresponding to the individual bite profile from a set of different pre-fabricated mouth guards are possible.

Alternative configurations of computing system may be used to implement process 900. For example, process 900 may be implemented on a mobile device having similar features described above, or on a retail kiosk residing in a bricks and mortar store.

Preferably, the sizing of mouth guards of various sizes made available for selecting in a retail environment, and the positioning and sizing of the pre-formed tooth-receiving indentations for each size are informed by a study of actual mouths. For example, a collection of digital models formed from a number of actual digital scans of individual mouths may be processed in order to establish a range of, for example, 10 sizes, with each mouth guard size in the range having pre-formed tooth-receiving indentations that would accommodate any wearer within the size subrange. In this way, a mouth guard for each subrange in the overall range or sizes can be constructed such that a wearer whose mouth size and configuration falls within a particular subrange can select a corresponding mouth guard with pre-formed tooth indentations. Generally speaking, the appropriate mouth guard for a particular wearer is one having a conformable component that is displaced, as compared to mouth guards sized according to other subranges in the overall range, the least to conform to the wearer's actual bite.

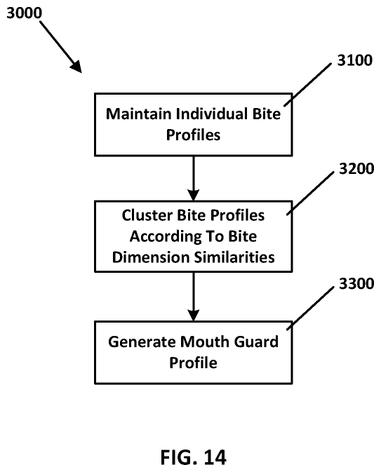

FIG. 14 is a flowchart depicting steps in a computer-implemented method 3000 for determining bite dimensions for each of a plurality of mouth guards, according to an embodiment. This method is useful for deriving the sizing and shaping of the mouth guards to be offered in a retail environment as pre-imprinted mouth guards, from actual bite profiles of individuals that have been received as a result of scanning for custom mouth guards. In this way, the actual bite profiles can inform the dimensions of mouth guards so that they are likely to properly accommodate bite profiles of individuals that have not been scanned for custom mouth guards.

Computer-implemented method 3000 and the relevant data being maintained for its execution may be executed on and hosted in whole or in part by a special purpose computer system such as computing system 1000. The computing system on which method 3000 is executed will generally not be the computing system on which method 900 is executed. Rather, the results of method 3000 will be made available on computing systems that are to implement the retail recommendation process of which method 900 is a part.

During method 3000, a plurality of individual bite profiles each comprising bite dimensions for respective individuals is maintained (step 3100). The individual bite profiles may number in the hundreds, the thousands, or the hundreds of thousands, and they may be pre-categorized according to race/ethnicity thereby to enable subsequent clustering, as will be described, to operate within the pre-categorizations. This may provide more meaningful clustering and resultant mouth guard profiles for each of a number of races/ethnicities rather than clustering across what could be significant different geometries resulting in less meaningful clustering, as will be described in more detail below. This bite profile data may be maintained in a structured relational database or other data structure and additional bite profile data may be added over time and used for re-generating more and more applicable mouth guard profiles, as such data is received from actual individuals having custom mouth guard scans conducted.

The individual bite profiles are clustered into a predetermined number of clusters according to similarities between respective bite dimensions (step 3200). For example, the bite profiles having the smallest arch dimensions are clustered in a different cluster than the bite profiles having the largest arch dimensions. Pursuant to this, for each of the clusters, based on the bite dimensions of the individual bite profiles in the cluster, at least one of a unique range of potential bite dimensions to be accommodated by a respective mouth guard; and unique representative bite dimensions for the respective mouth guard, is generated (step 3300) thereby to generate a mouth guard profile.

It is preferred that the predetermined number of clusters is at least four, thereby to provide more choice in the retail environment than is currently typically available. More preferably, the predetermined number of clusters is at least ten thereby to provide a good and useful range of quasi-custom sizing to the retail customers. In one embodiment, the number of clusters, and therefore resultant mouth guards, is 100. Alternatively, there are fewer clusters of actual sizes, but multiple different mouth guards available at each size for meeting different needs as specified by the purchaser filling out a questionnaire when selecting and purchasing, such as for higher or lower impact sports, for medical purposes (such as for night time sleeping), for accommodating dental appliances such as orthodontic brackets, for accommodating lower anterior teeth crowding full adult dentition, for large expanded arches, for lower collapsed arches, for pre-bite undersides to accommodate the individual's lower arch positioning with respect to the mouth guard, for missing adult pre-molars, and the like.

Because the specification of each mouth guard in the overall range is done by accounting for the shapes and sizes of actual mouths corresponding to a subrange of arch sizes and/or shapes, which of the mouth guards in the overall range is most appropriate to a particular wearer can be determined simply by measuring the arch size and/or shape of the wearer, or some proxy thereof.

In one embodiment, the established mouth guard size subranges do not overlap such that only one mouth guard size is appropriate for a given customer. However, it has been observed that a given customer may be found to have an arch size that corresponds exactly to an end point of one subrange of sizes and the start point of the next larger subrange of sizes. In this situation, the arch size of the customer may approach being nearly too large for one subrange while also being nearly too small for the next. As such, in an embodiment, adjacent subranges are defined to have a slight overlap with each other so that the customer whose arch size falls within the overlapping portions of the adjacent subranges can choose—or be recommended based on some criteria other than size such as age or intended primary use of the mouth guard—the mouth guard corresponding to the larger size or the mouth guard corresponding to the smaller size. For example, a child may be recommended the mouth guard corresponding to the larger size, since the child is may be inclined to grow out of the smaller size during the lifetime of the mouth guard or even within a given sports season. Similarly, a customer may be able to specify that he or she has a sensitivity or a particular mouth configuration in which case the customer may be recommended the smaller size.

The particular sizes of mouth guards according to the present invention that are made available in a particular retail environment may be selected, or even restricted, based on the distribution of sizes gleaned from actual mouth scans of buyers of custom mouth guards who are physically representative of those who the retailer has observed tends to live in the vicinity of the particular retail environment. For example, it has been suggested that a person's dentition roughly correlates with others sharing that person's race/ethnicity or sex, on average, as compared to others not sharing that person's race/ethnicity or sex (see, for example, “Influence of sex and ethnic tooth-size differences on mixed-dentition space analysis”, Altherr et al., Am J Orthod Dentofacial Orthop. 2007 September; 132(3): 332-339). As certain retail environments may observe they are being frequented more, on average, by those sharing a particular race/ethnicity as compared to other races/ethnicities or sex because of their geographic locations or aesthetic appeals, it may be extremely useful to those customers to be provided with a selection of mouth guards that have been pre-configured using prior scans of people who are actually physically similar to them, since a better mouth guard fit is likely to result. In an embodiment, therefore, in a master database the race/ethnicity and sex, to the extent it is appropriate and authorized, is collected and associated with the scans themselves such that ranges and subranges can, if desired, be established that better fit the expected customer than would a more generalized mouth guard that has been defined according to all of the scans in the master database.

It will be appreciate that the embodiments disclosed herein can be useful not just for protection during sports or other physical activity, but also for other activities such as for night-time sleeping, in order to ameliorate the effects of bruxism—otherwise known as involuntary grinding of the teeth that can occur when sleeping.

Although embodiments have been described with reference to the drawings, those of skill in the art will appreciate that variations and modifications may be made without departing from the spirit, scope and purpose of the invention as defined by the appended claims.

For example, in an embodiment, an electronic circuit is integrated within a gel protective pack integrated into the structural component of the mouth guard. The electronic circuit comprises RFID components for storing and communicating an unique identification of the mouth guard and, by extension, the individual wearing the mouth guard. Such identification may be suitable use in identifying the wearer and distinguishing the wearer from other contestants during time trials of a combines event or the like.

What is claimed is:

1. A mouth guard comprising:
a structural component having an arch portion including pre-formed tooth-receiving indentations; and
a conforming component supported by the structural component and being displaceable between the teeth of a wearer and the tooth-receiving indentations only when at or above a threshold temperature.

2. The mouth guard of claim 1, wherein the structural component and the conforming component are comprised of the same material, the density of the material in the structural component being greater than the density of the material in the conforming component.

3. The mouth guard of claim 1, wherein at least the conforming component is formed as a plurality of adjacent substantially planar surfaces intersecting along respective lines, wherein the lines soften and the surfaces wrap around a wearer's teeth when subjected to pressure by the teeth at or above the threshold temperature, thereby to conform to the wearer's teeth.

4. A computer-implemented method for recommending a mouth guard, the method comprising:
receiving electronic data including an individual bite profile comprising bite dimensions for an individual;
automatically selecting, from a set of different pre-fabricated mouth guards, at least one pre-fabricated mouth guard with a mouth guard profile most closely corresponding to the individual bite profile; and
generating an output message recommending the selected at least one pre-fabricated mouth guard.

5. The computer-implemented method of claim 4, comprising:
deriving the individual bite profile data from an electronic scan of the individual's mouth.

6. The computer-implemented method of claim 4, comprising:
deriving the individual bite profile data from one or more digital images of the individual's mouth.

7. The computer-implemented method of claim 4, comprising:
deriving the individual bite profile data from one or more digital images of bite paper used by the individual.

8. The computer-implemented method of claim 4, comprising:
deriving the individual bite profile data from one or more digital videos of the individual's mouth.

9. The computer-implemented method of claim 4, wherein the mouth guard profile for each of the pre-fabricated mouth guards in the set comprises a range of potential bite dimensions, wherein automatically selecting comprises calculating, using actual bite dimensions from the individual bite profile, within which of the ranges of potential bite dimensions the actual bite dimensions lies.

10. The computer-implemented method of claim 4, wherein the mouth guard profile for each of the pre-fabricated mouth guards in the set comprises representative bite dimensions, wherein automatically selecting comprises calculating, using actual bite dimensions from the individual bite profile, which of the representative bite dimensions differs the least from the actual bite dimensions.

11. The computer-implemented method of claim 8, further comprising:
providing an application for a mobile device, the application enabling a user to capture the one or more digital videos and provide the one or more digital videos for the deriving.

12. The computer-implemented method of claim 11, wherein the application comprises a user interface for aiding orientation of a digital video camera of the mobile device with respect to the mouth of the individual.

13. The computer-implemented method of claim 12, wherein the user interface comprises an augmented reality component for aiding the orientation of the digital video camera.

14. A computer-implemented method for determining bite dimensions for each of a plurality of mouth guards, the method comprising:
maintaining a plurality of individual bite profiles each comprising bite dimensions for respective individuals;
clustering the individual bite profiles into a predetermined number of clusters according to similarities between respective bite dimensions; and
for each of the clusters, generating, based on the bite dimensions of the individual bite profiles in the cluster, a mouth guard profile comprising at least one of:
a unique range of potential bite dimensions to be accommodated by a respective mouth guard; and
unique representative bite dimensions for the respective mouth guard.

15. The computer-implemented method of claim 14, wherein the predetermined number of clusters is at least four.

16. The computer-implemented method of claim 14, wherein the predetermined number of clusters is ten.

17. A processor-readable medium embodying a non-transitory computer program for recommending a mouth guard, the computer program comprising:
program code for receiving electronic data including an individual bite profile comprising bite dimensions for an individual;
program code for automatically selecting, from a set of different pre-fabricated mouth guards, at least one pre-fabricated mouth guard with a mouth guard profile most closely corresponding to the individual bite profile; and
program code for generating an output message recommending the selected at least one pre-fabricated mouth guard.

18. A computer-readable medium embodying a non-transitory computer program for determining bite dimensions for each of a plurality of mouth guards, the computer program comprising:
program code for maintaining a plurality of individual bite profiles each comprising bite dimensions for respective individuals;
program code for clustering the individual bite profiles into a predetermined number of clusters according to similarities between respective bite dimensions; and
program code for, for each of the clusters, generating, based on the bite dimensions of the individual bite profiles in the cluster, at least one of:
a unique range of potential bite dimensions to be accommodated by a respective mouth guard; and
unique representative bite dimensions for the respective mouth guard.

19. The mouth guard of claim 1, further comprising an electronic circuit integrated within a gel protective pack integrated into the structural component of the mouth guard, the electronic circuit comprising RFID components for storing and communicating an unique identification of the mouth guard thereby to identify the wearer.
